(19) United States
(12) Patent Application Publication
(10) Pub. No.: US 2011/0033941 A1
(43) Pub. Date: Feb. 10, 2011

(54) RISK ANALYSIS IN PATIENTS WITH AND WITHOUT METABOLIC SYNDROME

(76) Inventors: Georg Hess, Mainz (DE); Dietmar Zdunek, Tutzing (DE); Andrea Horsch, Mannheim (DE)

Correspondence Address: ROCHE DIAGNOSTICS OPERATIONS INC., 9115 Hague Road, Indianapolis, IN, 46250-0457, US

(21) Appl. No.: 12/908,921

(22) Filed: Oct. 21, 2010

Related U.S. Application Data
(63) Continuation of application No. PCT/EP2009/056082, filed on May 19, 2009.

(30) Foreign Application Priority Data
May 20, 2008 (EP) 08156545.9

(51) Int. Cl. G01N 33/48

(52) U.S. Cl. 436/86

(57) ABSTRACT

The present invention relates to a method for identifying a subject being susceptible to a metabolic syndrome related therapy based on determining the amounts of adiponectin, retinol binding protein 4, and proinsulin in a sample of a subject, and comparing the thus determined amounts to suitable reference amounts. Moreover, the present invention relates to a method for predicting the risk of developing a metabolic syndrome in an apparently healthy subject based on determining the aforementioned markers in a sample from the subject. Also encompassed by the present invention are kits and devices adapted to carry out the methods of the present invention.

### RELATED APPLICATIONS

This application is a continuation of international application PCT/EP2009/056082 filed May 19, 2009 and claims priority to European application EP 08156545.9 filed May 20, 2008.

### FIELD OF THE INVENTION

The present invention relates to a method for identifying a subject being susceptible to a metabolic syndrome related therapy based on determining the amounts of adiponectin, retinol binding protein 4 and proinsulin in a sample of a subject, and comparing the, thus, determined amounts to suitable reference amounts. Moreover, the present invention relates to a method for predicting the risk of developing a metabolic syndrome in an apparently healthy subject based on determining the aforementioned markers in a sample of said subject. Also encompassed by the present invention are kits and devices adapted to carry out the methods of the present invention.

### BACKGROUND OF THE INVENTION

The metabolic syndrome is associated with an imbalance between energy intake and the capacity for energy storage and results in the ectopic deposition of lipids in visceral fat, liver, skeletal muscle, pancreatic beta cells and vessel walls (Smith (2006) Obesity Vol. 14 Suppl. 128S-134S). It is a constellation of interrelated risk factors of metabolic origin that is considered to directly promote the development of atherosclerotic cardiovascular disease and diabetes type 2 (Grundy et al. (2005) Circulation 112, 2735-2752).

The predominant underlying risk factors for the syndrome are, presumably, abdominal obesity, insulin resistance. Other conditions that are associated with the metabolic syndrome can be physical inactivity and hormonal imbalances. The metabolic syndrome can occur both in obese and in non-obese patients. In non-obese patients, lipids are stored in visceral reservoirs. Once these reservoirs are filled, lipids are also stored in other tissues and organs. Especially, in the presence of visceral obesity, there is a significantly increased risk of progressing to diabetes type 2 and cardiovascular disease.

The diagnosis of the metabolic syndrome is difficult. Several organizations have attempted to formulate simple criteria for its diagnosis. The first proposal was made 1998 from a group preparing a definition for diabetes for the WHO. In this proposal, insulin resistance was seen as the major underlying risk factor and being required for diagnosis. This proposal followed the widely held belief that insulin resistance is the primary cause of the syndrome. A diagnosis according to the WHO criteria, thus, could be made when a patient exhibited one of several markers of insulin resistance plus two additional risk factors. Although insulin resistance is difficult to measure directly in a clinical setting, several types of indirect evidence were accepted such as impaired glucose intolerance, impaired fasting glucose, type 2 diabetes mellitus, or impaired disposal of glucose under hyperinsulinemic, euglycemic conditions. The additional risk factors used for diagnosis included obesity, hypertension, increased triglycerides, decreased HDL-C level, or the presence of microalbuminuria.

In 1999, the European Group for Study of Insulin Resistance (EGIR) suggested a modification of the WHO definition. The EGIR, similarly, suggested that insulin resistance is the major cause and, therefore, required evidence of it for diagnosis but focused more on abdominal obesity than did WHO. By their criteria, plasma insulin levels in the upper quartile of the population defined insulin resistance. Elevated plasma insulin plus two other factors—abdominal obesity, hypertension, elevated triglycerides or reduced HDL-C, and elevated plasma glucos—was proposed for the diagnosis. In contrast to the WHO criteria, the EGIR excluded patients with type 2 diabetes mellitus from their syndrome because insulin resistance was viewed primarily as a risk factor for diabetes (for a review for the various criteria see Grundy et al. (2005) Circulation 112, 2735-2752).

However, there is evidence that there are subjects that are at increased risk of developing diseases that are associated with the metabolic syndrome (cardiovascular diseases, tumors, diabetes, fatty liver disease) although not fulfilling the criteria for the metabolic syndrome. These subjects, therefore, require sufficient therapy. Moreover, there are subjects which have developed a metabolic syndrome (and, thus, fulfil the criteria), which are, however, not at increased risk of developing the aforementioned diseases. Such subjects may be treated too excessively resulting in increased health care costs.

Therefore, there is a need for a reliable identification of subject being susceptible to a metabolic syndrome related therapy, and there is a need for general risk stratification including the risk for developing a metabolic syndrome and diseases associated with a metabolic syndrome.

The technical problem underlying the present invention can be seen as the provision of means and methods for complying with the aforementioned needs.

The technical problem is solved by the embodiments characterized in the claims and herein below.

### SUMMARY OF THE INVENTION

Accordingly, the present invention relates to method for identifying a subject being susceptible to a metabolic syndrome related therapy, comprising the steps of
a) determining the amounts of adiponectin, retinal binding protein 4 (RBP4) and proinsulin in a sample of said subject,
b) comparing the amounts as determined in step a) to suitable reference amounts for adiponectin, RBP4 and proinsulin, and
c) identifying a subject being susceptible to a metabolic syndrome related therapy.

The method of the present invention, preferably, is an in vitro method. Moreover, it may comprise steps in addition to those explicitly mentioned above. For example, further steps may relate to sample pre-treatments or evaluation of the results obtained by the method. The method of the present invention may be also used for monitoring, confirmation, and subclassification of the subject. The method may be carried out manually or assisted by automation. Preferably, step (a), (b) and/or (c) may in total or in part be assisted by automation, e.g., by a suitable robotic and sensory equipment for the determination in step (a) or a computer-implemented comparison in step (b).

### BRIEF DESCRIPTION OF THE FIGURE

FIG. 1: High molecular weigh adiponectin, proinsulin and RBP4 in subjects with (n=356) and without (n=1907) the metabolic syndrome (MS).

### DETAILED DESCRIPTION OF THE INVENTION

The term “identifying” as used herein means assessing whether a subject will be susceptible and, thus, eligible to a metabolic syndrome related therapy or not. Particularly, subjects shall be identified which require said therapy and will, more particularly, benefit from said therapy. Preferably, a subject who is susceptible to said therapy will benefit from said therapy. A subject, preferably, benefits from a therapy, if the condition of said subject does not worsen or if the condition (particularly with respect to the metabolic syndrome) ameliorates. Preferably, a subject who is not susceptible to said therapy will not benefit from said therapy (e.g. said subject may have the risk of adverse side effects, high health care costs). As it will be understood by those skilled in the art, such an assessment is usually not intended to be correct for all (i.e. 100%) of the subjects to be identified. The term, however, requires that a statistically significant portion of subjects can be identified (e.g. a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. More preferably, at least 60%, at least 70%, at least 80% or at least 90% of the subjects of a population can be properly identified by the method of the present invention.

The term “subject” as used herein relates to animals, preferably mammals, and, more preferably, humans. It is contemplated by the present invention that said subject may be apparently healthy with respect to the metabolic syndrome. However, it is also contemplated that said subject already has developed a metabolic syndrome, and, thus, has the metabolic syndrome. The term “metabolic syndrome” is known in the art. As used herein the term, preferably, relates to a cluster of risk factors including hypertriglyceridemia, abdominal obesity, arterial hypertony, and various metabolic disorders including dyslipidaemia and hyperglycemia. In the art, different terms are known for the metabolic syndrome such as metabolic syndrome X, syndrome X, insulin resistance syndrome, and Reaven's syndrome. It is also known that various criteria exist for identifying individuals having the metabolic syndrome. Preferably, the metabolic syndrome as used herein is defined by the criteria according to the WHO (World Health Organization), or by the criteria according to the EGIR (European Group for the Study of Insulin Resistance), or by the criteria according to the NECP (National Education Program Adult Treatment Panel III, frequently also referred to as ATP III criteria) or by the AHA/NHLBI criteria (American Heart Association/Updated NCEP). Of these the EGIR criteria are more preferred, and the ATP III criteria are most preferred for the diagnosis of a metabolic syndrome. The various criteria defined by the said organizations are known in the art (see, for a review Scott M. Grundy et al., Circulation; 2005(112):2735-2752 which hereby is incorporated by reference in its entirety with respect to the disclosure content). In the context of the present invention, a subject who fulfils the specific criteria (see below) suffers from the metabolic syndrome, i.e. has developed a metabolic syndrome. Accordingly, a subject who does not fulfil said criteria has not developed the metabolic syndrome and, therefore, does not suffer from the metabolic syndrome.

The criteria according to the WHO (1998) require, for the diagnosis of a metabolic syndrome in a subject, the presence of insulin resistance identified by one of the following: diabetes mellitus, impaired glucose tolerance, impaired fasting glucose or for those with normal fasting glucose levels (<110 mg/dL) glucose uptake below the lowest quartile for background population under investigation under hyperinsulinemic, euglycemic conditions. In addition to insulin resistance, the presence at least two of the following is required:
a) blood pressure: ≧140 mmHg systolic or ≧90 mmHg diastolic (or on treatment for high blood pressure)
b) plasma triglycerides (TG): ≧1.695 mmol/L and high-density lipoprotein cholesterol (HDL C)≧0.9 mmol/L (for male subjects), ≧1.0 mmol/L (for female subjects)
c) waist to hip ratio >0.90 (for male subjects); >0.85 (for female subjects), and/or body mass index (BMI) >30 kg/m²
d) urinary albumin excretion ratio ≧20 μg/min or albumin to creatinine ratio ≧30 mg/g.

The US National Cholesterol Education Program Adult Treatment Panel III (ATP-III/NCEP, 2001) requires, for the presence/diagnosis of a metabolic syndrome, at least three of the following:
a) abdominal obesity given as waist circumference ≧102 cm (for male subjects), ≧88 cm (for female subjects)
b) plasma triglycerides ≧1.695 mmol/L (150 mg/dl)
c) HDL-C <40 mg/dL (for male subjects), <50 mg/dL (for female subjects)
d) blood pressure ≧130/≧85 mmHg
e) fasting plasma glucose ≧6.1 mmol/L (110 mg/dl)

The European Group for the Study of Insulin Resistance (EGIR) requires, for the diagnosis/presence of a metabolic syndrome, insulin resistance defined as the top 25% of the fasting insulin values among non-diabetic individuals, and two or more of the following:
a) central obesity: waist circumference ≧94 cm (male), ≧80 cm (female) and/or body mass index (BMI) ≧30 kg/m²
b) plasma triglycerides ≧150 mg/dL and/or HDL-C <39 mg/dL (or treated for dyslipidaemia)
c) hypertension: blood pressure ≧140/90 mm Hg (or antihypertensive medication)
d) fasting plasma glucose ≧6.1 mmol/L

The AHA/NHLBI criteria American Heart Association/Updated NCEP require, for the presence/diagnosis of a metabolic syndrome, at least three of the following: elevated waist circumference: (Men: Equal to or greater than 102 cm, women equal to or greater than 88 cm); elevated triglycerides (equal to or greater than 150 mg/dl); reduced HDL cholesterol (men: less than 40 mg/dL; women: less than 50 mg/dL); elevated blood pressure (equal to or greater than 130/85 mm Hg or use of medication for hypertension) elevated fasting glucose (equal to or greater than 100 mg/dL (5.6 mmol/L) or use of medication for hyperglycemia).

It is to be understood that the metabolic syndrome may be already diagnosed or not diagnosed.

“A subject who is apparently healthy with respect to the metabolic syndrome”, preferably, is a subject who has not developed the metabolic syndrome, and, thus, does not fulfil the criteria, preferably, according to the WHO, more preferably according to the EGIR, and, most preferably, according to the NCEP (ATP III) for the presence of the metabolic syndrome as indicated herein above. Thus, the subject shall not have a metabolic syndrome.

The term “metabolic syndrome related therapy”, preferably, encompasses those treatment regimens intended to ameliorate and to treat the metabolic syndrome. Such treatment regimens are known in the art. Also contemplated are treatment regimens intended to prevent the development of a metabolic syndrome. Preferably, the treatment regimes include lifestyle changes and the administration of pharmaceuticals. However, also contemplated are surgery treatment regimens that allow weight reduction, preferably, placement of a gastric band (also known as Laparoscopic Adjustable Gastric Banding). Preferred lifestyle changes in the context of the present invention are regular exercise, caloric restriction, weight reduction, reduction of sodium intake, reduction of alcohol consumption, and smoking cessation. Particularly, subjects with overweight (and low adiponectin amounts) shall focus on losing weight.

Preferably, administered pharmaceuticals are pharmaceuticals that allow treating the individual disorders comprised by the metabolic syndrome such as hyperlipidemia, hypertension and high blood glucose. Said pharmaceuticals are known in the art. Preferred pharmaceuticals to be administered to a subject being susceptible to a metabolic syndrome related therapy are selected from the group consisting of lipid-lowering pharmaceuticals (which, preferably, aim to treat hyperlipidemia), β-adrenergic blockers, diuretics, ACE inhibitors, angiotensin II receptor blockers, calcium channel blockers (which, preferably, aim to treat hypertension), insulin sensitizers such as metformin hydrochloride and thiazolidinedones (such as glitazone, medione, pioglitazone, rosiglitazone, troglitazone) which, preferably, aim to treat high blood glucose), and aldosterone antagonists, preferably, spironolactone and eplerenone. Other pharmaceutical that are, preferably, administered to a subject being susceptible to a metabolic syndrome related therapy are alpha glucosidase inhibitors, angiotensin II type 1 receptor blockers, DPP-IV inhibitors (Dipeptidyl peptidase IV inhibitors) and endocannabinoids.

Lipid-lowering pharmaceuticals are known to the person skilled in the art. Examples include fibrates (e.g. bezofibrate, clofibrate, etofibrate, etophylline clofibrate, fenofibrate, gemfibrozil), nicotinic acid and analogs thereof (e.g. nicotinic acid, acipimox), statins (e.g. simvastatin, lovastatin, pravastatin, fluvastatin, atorvastatin, cerivastatin), anion exchanger resins (e.g. colestyramine, colestipol), probucol, and sitosterol. Preferred lipid-lowering pharmaceuticals in the present context are statins.

ACE-inhibitors are known to the person skilled in the art. Examples include benazepril, captopril, cilazapril, enalapril, fosinopril, lisinopril, moexipril, perindopril, quinapril, ramipril, spirapril, and trandolapril.

β-adrenergic blockers (non-selective and β₁-selective) are known to the person skilled in the art. Examples include acebutolol, alprenolol, atenolol, betaxolol, bisoprolol, bupranolol, carazolol, carteolol, carvedilol, celiprolol, metipranolol, metoprolol, nadolol, nebivolol, oxprenolol, penbutolol, pindolol, propanolol, sotalol, tanilolol, and timolol.

The term “proinsulin” is well known in the art. As used herein, the term, preferably, relates to the single chain polypeptide that is the precursor of insulin. Proinsulin is synthesized in the pancreatic beta cells and then enzymatically cleaved, releasing insulin into the circulation together with the C-peptide which connects the A and B chains of insulin within the proinsulin molecule. The sequence of proinsulin is highly conserved in mammalian species and is homologous with IGF-1 and IGF-2. Preferably, the term “proinsulin” relates to human proinsulin. Preferably, the term proinsulin also includes variants of proinsulin. How determine the amount of proinsulin is well known in the art (see, e.g. Houssa P. et al.: First direct assay for intact human proinsulin. Clinical Chemistry, 44(7): 15414-1519 (1998); or Linde S. et al. Chromatogr., 548(1-2): 371-380 (1991)).

In the context of the present invention, it is also contemplated to determine the amount of the C-peptide instead of determining the amount of proinsulin.

Adiponectin is a polypeptide (one of several known adipocytokines) secreted by the adipocyte. Adiponectin exists in a wide range of multimer complexes in plasma and combines via its collagen domain to create 3 major oligomeric forms: a low-molecular weight (LMW) trimer, a middle-molecular weight (MMW) hexamer, and high-molecular weight (HMW) 12- to 18-mer adiponectin (Kadowaki et al.(2006) Adiponectin and adiponectin receptors in insulin resistance, diabetes, and the metabolic syndrome. J Clin Invest. 116(7): 1784-1792, Rexford S. Ahima, Obesity 2006; 14:242 S-249S). Adiponectin has been reported to have several physiological actions, such as protective activities against atherosclerosis, improvement of insulin sensitivity, and prevention of hepatic fibrosis.

Adiponectin as used herein, preferably, relates to low molecular weight adiponectin, mid molecular weight adiponectin, more preferably, to total adiponectin, and, most preferably, to high molecular weight adiponectin (HMW) adiponectin (12- to 18-mer adiponectin, preferably, 18-mer adiponectin). The terms high molecular weight adiponectin, mid molecular weight adiponectin and total adiponectin are understood by the skilled person (see Kadowaki, loc. cit. which is hereby incorporated by reference in its entirety). Preferably, said adiponectin is human adiponectin. Preferably, the term “adiponectin” also includes variants of adiponectin. How to determine the amount of the various adiponectins is well known in the art. Methods for the determination of adiponectin are, e.g., disclosed in US 2007/0042424 A1 as well as in WO/2008/084003. Moreover, the sequence of the adiponectin polypeptide is well known in the art, and is, e.g., disclosed in WO/2008/084003 which is herewith incorporated by reference in its entirety.

The term “retinol binding protein 4”, herein also referred to as RBP4 or as retinol binding protein, is well known to the skilled person. The term also encompasses variants of RBP4 Preferably, said term relates to human RBP4. RBP4 is a polypeptide and a member of the lipocalins and is capable of binding to retinol. Preferably, the term retinol binding protein 4 also includes variants of the retinol binding protein. Specifically, RBP4 delivers retinol from the liver to the peripheral tissues. The sequence of RBP4 as well as methods for determining the amount of RBP4 are well known in the art (for the sequence, e.g., see Genbank Acc. Number NM_{—}006744.3 (nucleic acid sequence)/NP_{—}006735.2 (protein sequence))

A variant in the context of the present invention, preferably, has at least the same essential biological and immunological properties as the specific polypeptide (RBP4, proinsulin, adiponectin, a natriuretic peptide and CRP respectively). In particular, they share the same essential biological and immunological properties if they are detectable by the same specific assays referred to in this specification, e.g., by ELISA assays using polyclonal or monoclonal antibodies specifically recognizing the said specific polypeptides. A preferred assay is described in the accompanying Examples. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific polypeptides. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85:2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs. Moreover, the variants referred to herein include fragments of the specific polypeptides or the aforementioned types of variants as long as these fragments have the essential immunological and biological properties as referred to above. Such fragments may be, e.g., degradation products of the polypeptides. Further included are variants which differ due to posttranslational modifications such as phosphorylation or myristylation.

Preferably, the polypeptides referred to in the context of the present invention relate to human polypeptides.

The term “sample” refers to a sample of a body fluid, to a sample of separated cells or to a sample from a tissue or an organ. Samples of body fluids can be obtained by well known techniques and include, preferably, samples of blood, plasma, serum, or urine, more preferably, samples of blood, plasma or serum. Most preferably, the sample is a serum. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from those cells, tissues or organs which express or produce the peptides referred to herein.

Determining the amount of RBP4 (retinol binding protein 4), proinsulin, adinponectin, or of any other peptide or polypeptide referred to in this specification relates to measuring the amount or concentration, preferably semi-quantitatively or quantitatively. Measuring can be done directly or indirectly. Direct measuring relates to measuring the amount or concentration of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e. a component not being the peptide or polypeptide itself) or a biological read out system, e.g., measurable cellular responses, ligands, labels, or enzymatic reaction products.

In accordance with the present invention, determining the amount of a peptide or polypeptide can be achieved by all known means for determining the amount of a peptide in a sample. Said means comprise immunoassay devices and methods which may utilize labeled molecules in various sandwich, competition, or other assay formats. Said assays will develop a signal which is indicative for the presence or absence of the peptide or polypeptide. Moreover, the signal strength can, preferably, be correlated directly or indirectly (e.g. reverse-proportional) to the amount of polypeptide present in a sample. Further suitable methods comprise measuring a physical or chemical property specific for the peptide or polypeptide such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on Elecsys analyzers), CBA (an enzymatic cobalt binding assay, available for example on Roche-Hitachi analyzers), and latex agglutination assays (available for example on Roche-Hitachi analyzers).

Preferably, determining the amount of a peptide or polypeptide comprises the steps of (a) contacting a cell capable of eliciting a cellular response the intensity of which is indicative of the amount of the peptide or polypeptide with the said peptide or polypeptide for an adequate period of time, (b) measuring the cellular response. For measuring cellular responses, the sample or processed sample is, preferably, added to a cell culture and an internal or external cellular response is measured. The cellular response may include the measurable expression of a reporter gene or the secretion of a substance, e.g. a peptide, polypeptide, or a small molecule. The expression or substance shall generate an intensity signal which correlates to the amount of the peptide or polypeptide.

Also preferably, determining the amount of a peptide or polypeptide comprises the step of measuring a specific intensity signal obtainable from the peptide or polypeptide in the sample. As described above, such a signal may be the signal intensity observed at an m/z variable specific for the peptide or polypeptide observed in mass spectra or a NMR spectrum specific for the peptide or polypeptide.

Determining the amount of a peptide or polypeptide may, preferably, comprises the steps of (a) contacting the peptide with a specific ligand, (b) (optionally) removing non-bound ligand, (c) measuring the amount of bound ligand. The bound ligand will generate an intensity signal. Binding according to the present invention includes both covalent and non-covalent binding. A ligand according to the present invention can be any compound, e.g., a peptide, polypeptide, nucleic acid, or small molecule, binding to the peptide or polypeptide described herein. Preferred ligands include antibodies, nucleic acids, peptides or polypeptides such as receptors or binding partners for the peptide or polypeptide and fragments thereof comprising the binding domains for the peptides, and aptamers, e.g. nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g. phage display. Antibodies as referred to herein include both polyclonal and monoclonal antibodies, as well as fragments thereof, such as Fv, Fab and F(ab)₂ fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present invention means that the ligand or agent should not bind substantially to (“cross-react” with) another peptide, polypeptide or substance present in the sample to be analyzed. Preferably, the specifically bound peptide or polypeptide should be bound with at least 3 times higher, more preferably at least 10 times higher and even more preferably at least 50 times higher affinity than any other relevant peptide or polypeptide. Non-specific binding may be tolerable, if it can still be distinguished and measured unequivocally, e.g. according to its size on a Western Blot, or by its relatively higher abundance in the sample. Binding of the ligand can be measured by any method known in the art. Preferably, said method is semi-quantitative or quantitative. Suitable methods are described in the following.

First, binding of a ligand may be measured directly, e.g. by NMR or surface plasmon resonance.

Second, if the ligand also serves as a substrate of an enzymatic activity of the peptide or polypeptide of interest, an enzymatic reaction product may be measured (e.g. the amount of a protease can be measured by measuring the amount of cleaved substrate, e.g. on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, preferably the amount of substrate is saturating. The substrate may also be labeled with a detectable lable prior to the reaction. Preferably, the sample is contacted with the substrate for an adequate period of time. An adequate period of time refers to the time necessary for an detectable, preferably measurable, amount of product to be produced. Instead of measuring the amount of product, the time necessary for appearance of a given (e.g. detectable) amount of product can be measured.

Third, the ligand may be coupled covalently or non-covalently to a label allowing detection and measurement of the ligand. Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star (Amersham Biosciences), ECF (Amersham Biosciences). A suitable enzyme-substrate combination may result in a colored reaction product, fluorescence or chemiluminescence, which can be measured according to methods known in the art (e.g. using a light-sensitive film or a suitable camera system). As for measuring the enzymatic reaction, the criteria given above apply analogously. Typical fluorescent labels include fluorescent proteins (such as GFP and its derivatives), Cy3, Cy5, Texas Red, Fluorescein, and the Alexa dyes (e.g. Alexa 568). Further fluorescent labels are available e.g. from Molecular Probes (Oregon). Also the use of quantum dots as fluorescent labels is contemplated. Typical radioactive labels include ³⁵S, ¹²⁵I, ³²P, ³³P and the like. A radioactive label can be detected by any method known and appropriate, e.g. a light-sensitive film or a phosphor imager. Suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamide gel electrophoresis (SDS-PAGE), Western Blotting, and mass spectrometry), can be used alone or in combination with labeling or other detection methods as described above.

The term “amount” as used herein encompasses the absolute amount of a polypeptide or peptide, the relative amount or concentration of the said polypeptide or peptide as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “comparing” as used herein encompasses comparing the amount of the peptide or polypeptide comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample. The comparison referred to in step (b) of the method of the present invention may be carried out manually or computer assisted. For a computer assisted comparison, the value of the determined amount may be compared to values corresponding to suitable references which are stored in a database by a computer program. The computer program may further evaluate the result of the comparison, i.e. automatically provide the desired assessment in a suitable output format. Based on the comparison of the amount determined in step a) and the reference amount, it is possible to assess whether a subject is susceptible for a metabolic syndrome related therapy, i.e. belonging to the group of subjects which can be successfully treated by the applying a metabolic syndrome related therapy. Therefore, the reference amount is to be chosen so that either a difference or a similarity in the compared amounts allows identifying those the test subject which belong into the group of subjects susceptible for metabolic syndrome related therapy or identifying those test subjects which are not susceptible for a metabolic syndrome related therapy.

Accordingly, the term “reference amount” as used herein refers to an amount which allows for identifying a subject being susceptible to a metabolic syndrome related therapy. Accordingly, the reference may either be derived from (i) a subject known to be susceptible to a metabolic syndrome related therapy, or (ii) a subject known not to be susceptible to a metabolic syndrome related therapy. Moreover, the reference amount for RBP4, proinsulin and for adiponectin, respectively, according to the invention may define a threshold amount for RBP4, proinsulin and for adiponectin, respectively, whereby an amount of RBP4, and proinsulin larger and an amount of adiponectin lower than the respective threshold shall be indicative for a subject being susceptible to a metabolic syndrome related therapy while an amount of RBP4, and proinsulin lower and an amount of adiponectin larger than the threshold amount shall be an indicator for a subject being not susceptible to a metabolic syndrome related therapy. The reference amount applicable for an individual subject may vary depending on various physiological parameters such as age, gender, or subpopulation, as well as on the means used for the determination of the polypeptide or peptide referred to herein. A suitable reference amount may be determined by the method of the present invention from a reference sample to be analyzed together, i.e. simultaneously or subsequently, with the test sample. A preferred reference amount serving as a threshold may be derived from the upper limit of normal (ULN), i.e. the upper limit of the physiological amount to be found in a population of subjects not requiring a metabolic syndrome related therapy. The ULN for a given population of subjects can be determined by various well known techniques. A suitable technique may be to determine the median of the population for the peptide or polypeptide amounts to be determined in the method of the present invention.

A reference amount defining a threshold amount for RBP4 as referred to in accordance with the present invention is, preferably, 47 mg/l, more preferably 41 mg/ml, and even more preferably, 43 mg/l.

A reference amount defining a threshold amount for proinsulin as referred to in accordance with the present invention is, preferably, 2.8 pmol/l, more preferably 2.0 pmol/l, and even more preferably, 2.2 pmol/l.

A reference amount defining a threshold amount for adiponectin, particularly, for high molecular weight adiponectin, as referred to in accordance with the present invention is, preferably, 2.0 μg/ml, more preferably, 2.4 μg/ml and even more preferably, 2.8 μg/ml, or 3.0 μg/ml.

An amount of adiponectin lower than the reference amount and an amount of RBP4 and proinsulin larger than the reference amounts for RBP4 and a proinsulin is, preferably, indicative for a subject being susceptible to a metabolic syndrome related therapy.

An amount of adiponectin larger than the reference amount and an amount of RBP4 and proinsulin lower than the reference amounts for RBP4 and proinsulin is, preferably, indicative for a subject not being susceptible to a metabolic syndrome related therapy. Of course, said patient still may benefit from lifestyle changes that are indicated herein above.

Subjects who do not fulfil the criteria above ((a) adiponectin lower than reference, RPB4 and proinsulin larger than the reference amount, or (b) adiponectin larger than reference, RPB4 and proinsulin lower than the reference amount) should be carefully monitored in order to determine whether they are susceptible to a metabolic syndrome related therapy. E.g. the markers shall be redetermined within certain intervals, preferably intervals of 6 months or 1 year.

Advantageously, it has been found in the studies underlying the present invention that determining the amounts of adiponectin, RBP4, and proinsulin is required for reliably identifying a subject being susceptible to a metabolic syndrome related therapy. Since it is very difficult to assess the metabolic syndrome (a variety of different criteria have been proposed) and to assess the risk thereof, it is also difficult to identify subjects that require a therapy related to the metabolic syndrome.

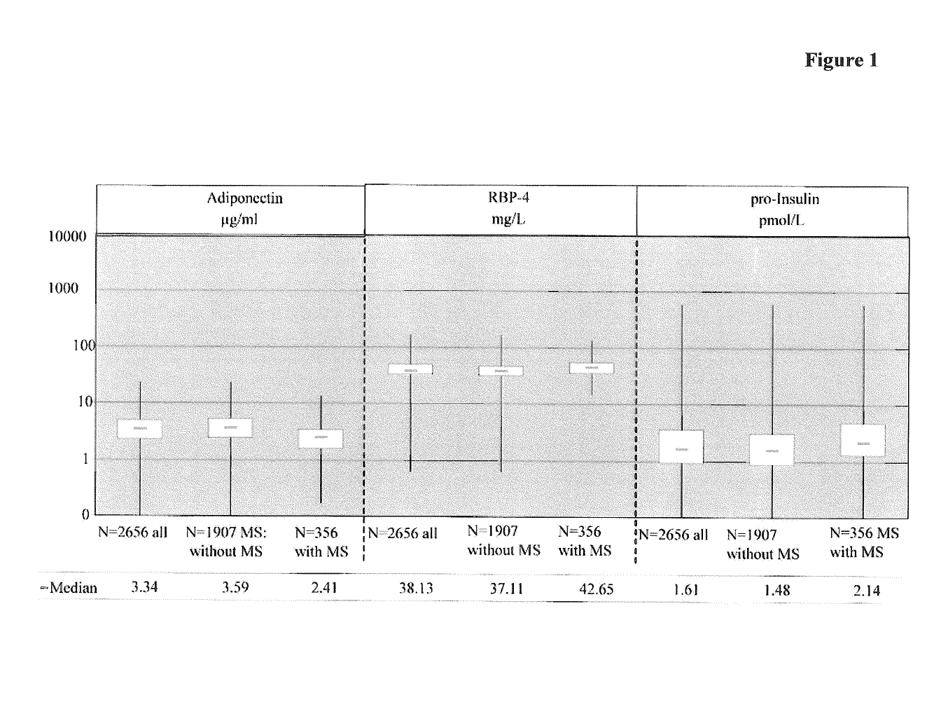

In the context of the present invention, various markers (RBP4, high molecular weight adiponectin, proinsulin, NT-proBNP and CRP) were determined in subjects with and without the metabolic syndrome (see Examples). Generally, the amounts of proinsulin and RBP4 (and CRP and NT-proBNP) were larger in samples of individuals with the metabolic syndrome than in samples individuals without the metabolic syndrome, and the amount of adiponectin was lower in samples of individuals with the metabolic syndrome than in samples individuals without the metabolic syndrome (see FIG. 1).

Interestingly, a portion of individuals without the metabolic syndrome, however, had elevated proinsulin and RBP4 amounts and a reduced adiponectin amount indicating the need for a metabolic therapy. These are patients which will benefit from a metabolic syndrome related therapy since they are at risk of developing the metabolic syndrome and/or of suffering from a sequela thereof. Moreover, there were patients with the metabolic syndrome which did not show an increase of amounts proinsulin and RBP4 and did not show a decrease of adiponectin. These are patients that will be overtreated with a therapy related to the metabolic syndrome (resulting, e.g., in increased health care costs).

Thus, it was found that not all patients that have developed a metabolic syndrome necessarily require a metabolic syndrome related therapy (herein referred to a low risk patients), whereas some patients that are apparently healthy with respect to the metabolic syndrome (patients which do not meet the criteria of a metabolic syndrome), will benefit from a metabolic syndrome related therapy (herein referred to as high risk patients, thus patients which particularly are at elevated risk of developing cardiovascular diseases/disorders, diabetes, tumors in the future).

The present invention will be, if applied, very beneficial to the health care system, since a) it allows to identify subjects that are susceptible to a metabolic syndrome related therapy which would, by methods of the prior art, not have been identified as being susceptible to a metabolic syndrome related therapy (particularly subjects that are apparently healthy with respect to a metabolic syndrome, the subjects of the high risk group) and b) to identify subjects with a metabolic syndrome not being susceptible to a metabolic syndrome related therapy which might previously been treated to much, (particularly subjects of the so called low risk group).

Thus, in case the patient turns out to be not susceptible for a certain therapy, a time and/or cost intensive therapy can be avoided. Thus, besides preventing a subject from the adverse and side effects that may accompany a metabolic syndrome related therapy, the method of the present invention will be beneficial for the health system in that resources will be saved.

Preferably, the method of the present invention further comprises determining the amount of a natriuretic peptide and/or CRP(C-reactive protein) in a sample (preferably, a blood, blood serum or a blood plasma sample) of the subject and comparing the, thus, determined amount of a natriuretic peptide to a suitable reference amount for said natriuretic peptide and/or amount of CRP. Said amounts may be determined in the same sample in which the amount of the other polypeptides referred to herein is/was determined or in a different sample (e.g. a second or a third sample). Also contemplated is simultaneous determination of RBP4, proinsulin, Adiponectin, said natriuretic peptide and CRP.

The determination of the additional markers referred to above will, preferably, increase the sensitivity and specificity of the identification of subjects susceptible to a metabolic syndrome related therapy.

Natriuretic peptides according to the present invention comprise ANP-type and BNP-type peptides and variants thereof (see e.g. Bonow, R. O. (1996). New insights into the cardiac natriuretic peptides. Circulation 93: 1946-1950).

ANP-type peptides comprise pre-proANP, proANP, NT-proANP, ANP, and variants thereof.

BNP-type peptides comprise pre-proBNP, proBNP, NT-proBNP, BNP, and variants thereof.

The pre-pro peptide (134 amino acids in the case of pre-proBNP) comprises a short signal peptide, which is enzymatically cleaved off to release the pro peptide (108 amino acids in the case of proBNP). The pro peptide is further cleaved into an N-terminal pro peptide (NT-pro peptide, 76 amino acids in case of NT-proBNP) and the active hormone (32 amino acids in the case of BNP, 28 amino acids in the case of ANP).

Preferred natriuretic peptides according to the present invention are NT-proANP, ANP, NT-proBNP, BNP, and variants thereof. ANP and BNP are the active hormones and have a shorter half-life than their respective inactive counterparts, NT-proANP and NT-proBNP. Therefore, depending on the time-course that is of interest, either measurement of the active or the inactive forms can be advantageous. The most preferred natriuretic peptides according to the present invention are BNP-type peptides and variants thereof, particularly NT-proBNP and variants thereof.

CRP, herein also referred to as C-reactive protein, is an acute phase protein that was discovered more than 75 years ago to be a blood protein that binds to the C-polysaccharide of pneumococci. CRP is known as a reactive inflammatory marker and is produced by a distal organ (i.e. the liver) in response or reaction to chemokines or interleukins originating from the primary lesion site. CRP consists of five single subunits, which are non covalently linked and assembled as a cyclic pentamer with a molecular weight of approximately 110-140 kDa. Preferably, CRP as used herein relates to human CRP. The sequence of human CRP is well known and disclosed, e.g., by Woo et al. (J. Biol. Chem. 1985. 260 (24), 13384-13388). The level of CRP is usually low in normal individuals but can rise 100- to 200-fold or higher due to inflammation, infection or injury (Yeh (2004) Circulation. 2004; 109:II-11-II-14). It is known that CRP is an independent factor for the prediction of a cardiovascular risk. Particularly, it has been shown that CRP is suitable as a predictor for myocardial infarction, stroke, peripheral arterial disease and sudden cardiac death. Moreover, elevated CRP amounts may also predict recurrent ischemia and death in subjects with acute coronary syndrome (ACS) and those undergoing coronary intervention. Determination of CRP is recommended by expert panels (e.g. by the American Heart Association) in patients with a risk of coronary heart disease (see also Pearson et al. (2003) Markers of Inflammation and Cardiovascular Disease. Circulation, 107: 499-511). The term CRP also relates to variants thereof.

Preferably, the amount of CRP in a sample of a subject is determined by using CRP assays with a high sensitivity. The CRP determined by such assays is frequently also referred to as high sensitivity CRP (hsCRP). hsCRP assays are, e.g., used to predict the risk of heart disease. Suitable hsCRP assays are known in the art. A particularly preferred hsCRP assay in the context of the present invention is the Roche/Hitachi CRP (Latex) HS test with a detection limit of 0.1 mg/l.

A reference amount for a natriuretic peptide may be obtained as described earlier in this specification. A reference amount defining a threshold amount for a natriuretic peptide, particularly, for NT-proBNP, as referred to in accordance with the present invention is, preferably, 70 pg/ml, more preferably, 120 pg/ml, and, most preferably, 100 pg/ml.

A reference amount for CRP may be obtained as described earlier in this specification. A reference amount defining a threshold amount for CRP as referred to in accordance with the present invention is, preferably, 3.0 mg/l, more preferably, 4.0 mg/l and, most preferably, 5.0 mg/l.

Determining the amount of a natriuretic peptide and/or CRP allows further classification of a subject and, therefore, to make further decisions on certain treatment regimens.

Preferably, an amount of a natriuretic peptide, particularly of NT-proBNP, and/or CRP larger than the reference amount (for a natriuretic peptide and/or CRP, if both the natriuretic peptide and CRP are determined: larger than the reference amount for a natriuretic peptide and CRP) further indicates/confirms that said subject is susceptible to a metabolic syndrome related therapy provided that the amounts of RBP4, proinsulin and adiponectin also indicate that said subject is susceptible to a metabolic syndrome related therapy (see above). It is to be understood that, in this case, for an amount of a natriuretic peptide larger than the reference, one focus of the treatment, preferably, will be administration of pharmaceuticals that are suitable for treating heart diseases and disorders, preferably ACE-inhibitors, statins and β-adrenergic blockers. Preferably, if the amount of CRP is larger than the reference, one focus of treatment will be medication with, preferably, anti-inflammatory drugs such as NSAIDs, and/or with lipid-lowering drugs (e.g. statins).

Preferably, an amount of a natriuretic peptide, particularly of NT-proBNP, and/or CRP lower than the reference amount further indicates/confirms that said subject is not susceptible to a metabolic syndrome related therapy provided that the amounts of RBP4, proinsulin and adiponectin also indicate that said subject is not susceptible to a metabolic syndrome related therapy.

In a preferred embodiment of the method of the present invention, the method is for identifying a subject being susceptible to a metabolic syndrome related therapy, comprising the steps of
(a) determining the amounts of adiponectin, retinol binding protein 4 (RBP4) and proinsulin in a sample of said subject,
(b) comparing the amounts as determined in step a) to suitable reference amounts for adiponectin, RBP4 and proinsulin, and
(c) identifying a subject being susceptible to a metabolic syndrome related therapy,
   wherein said subject is apparently healthy with respect to the metabolic syndrome, and wherein (i) an amount of adiponectin lower than the reference amount for adiponectin, (ii) an amount of RBP4 larger than the reference amount for RBP4, and (iii) an amount of proinsulin larger than the reference amount for proinsulin indicates that said subject is susceptible to a metabolic syndrome related therapy.

As laid out herein above, said method may also comprise the determination of a natriuretic peptide and/or CRP.

In another preferred embodiment of the method of the present invention, the method allows identifying a subject not being susceptible to a metabolic syndrome related therapy, comprising the steps of
(b) determining the amounts of adiponectin, retinol binding protein 4 (RBP4) and proinsulin in a sample of said subject,
(d) comparing the amounts as determined in step a) to suitable reference amounts for adiponectin, RBP4 and proinsulin, and
(e) identifying a subject not being susceptible to a metabolic syndrome related therapy,
   wherein said subject already has developed a metabolic syndrome, and wherein an amount of adiponectin larger than the reference amount for adiponectin, (ii) an amount of RBP4 lower than the reference amount for RBP4, and (iii) an amount of proinsulin lower than the reference amount for proinsulin indicates that said subject is not susceptible to a metabolic syndrome related therapy.

As laid out in this specification, the aforementioned method may further comprise the determination a natriuretic peptide and/or CRP.

The definitions given above apply mutatis mutandis to the following.

Furthermore, the present invention relates to a method for predicting, in an apparently healthy subject, the risk of developing a metabolic syndrome (and/or the risk of developing a sequela of the metabolic syndrome selected from the group consisting of tumor diseases, cardiovascular diseases, diabetes and fatty liver disease), comprising the steps of
(a) determining the amounts of adiponectin, retinol binding protein (RBP4) and proinsulin in a sample of said subject;
(b) comparing the amounts as determined in step a) to suitable reference amounts for adiponectin, RBP4 and proinsulin; and
(c) predicting the risk of developing a metabolic syndrome (and/or of developing the said sequela of the metabolic syndrome).

The term “predicting” as used to assessing the probability according to which a subject which is apparently healthy with respect to a metabolic syndrome will develop a metabolic syndrome (and/or of developing the said sequela) within a defined time window (predictive window) in the future. The predictive window is an interval in which the subject will develop a metabolic syndrome. The predictive window may be the entire remaining lifespan of the subject upon analysis by the method of the present invention. Preferably, however, the predictive window is an interval of six months or one, two, three, four, five or ten years after carrying out the method of the present invention (more preferably and precisely, after the sample to be analyzed by the method of the present invention has been obtained). As will be understood by those skilled in the art, such an assessment is usually not intended to be correct for 100% of the subjects to be analyzed. The term, however, requires that the assessment will be valid for a statistically significant portion of the subjects to be analyzed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the prediction will be correct for at least 60%, at least 70%, at least 80%, or at least 90% of the subjects of a given cohort.

The term “predicting the risk of developing a metabolic syndrome” as used herein means that the subject to be analyzed by the method of the present invention is allocated either into the group of subjects of a population having a normal, i.e. non-elevated, risk for developing a metabolic syndrome or into a group of subjects having a significantly elevated risk. An elevated risk as referred to in accordance with the present invention also means that the risk of developing a metabolic syndrome within a predetermined predictive window is elevated significantly for a subject with respect to the average risk for a metabolic syndrome of subjects as defined herein. It is to be understood that average risk may vary depending on age and gender. Preferably, for a predictive window of three years, the average risk is within the range of 0.5 and 3.0%, preferably, 2.0%. An elevated risk as used herein, preferably, relates to a risk of more than 3.0, preferably, more than 5.0%, even more preferably, more than 10%, and, most preferably within 3.0% and 15%, or even more than 15%, with respect to a predictive window of three years. The given risks, preferably, are based on a cohort of adult subjects, preferably subjects older than 20 years.

Preferred reference amounts for the markers RBP4, proinsulin, and adiponectin are given herein above.

Preferably, (i) an amount of adiponectin lower than the reference amount for adiponectin, (ii) an amount of RBP4 larger than the reference amount for RBP4, and (iii) an amount of proinsulin larger than the reference amount for proinsulin indicates that the subject is at risk of developing a metabolic syndrome (and/or of developing the said sequela).

Preferably, (i) an amount of adiponectin larger than the reference amount for adiponectin, (ii) an amount of RBP4 lower than the reference amount for RBP4, and (iii) an amount of proinsulin lower than the reference amount for proinsulin indicates that the subject is not at risk of developing a metabolic syndrome (and/or of developing the said sequela).

Other subjects who do not fulfil the criteria above ((a) adiponectin lower than reference, RPB4 and proinsulin larger than reference amount or (b) adiponectin larger than reference, RPB4 and proinsulin lower than reference amount), preferably, should be carefully monitored to predict the risk (see also above).

In a preferred embodiment, the aforementioned method of the present invention further comprises the determination of the amount of a natriuretic peptide, preferably of NT-proBNP, and/or of CRP (C-reactive protein) in a sample of the respective subject and the comparison of said amount to a suitable reference amount. Preferred reference amounts for a natriuretic peptide and CRP are given herein above. Preferably, an amount of a natriuretic peptide and/or CRP larger than the respective reference amount further indicates/confirms that said subject is at risk/at increased risk of developing a metabolic syndrome (provided that also the amount of the other determined markers indicate that said subject is at risk), whereas an amount of a natriuretic peptide and/or CRP lower than the reference amount further indicates/confirms that said subject is not at risk of developing a metabolic syndrome.

It is to be understood that a subject who is at risk of developing a metabolic syndrome also is at risk of developing diseases that are frequently linked to the metabolic syndrome (sequela of the metabolic syndrome), preferably, cardiovascular diseases, diabetes, particularly type 2 diabetes, tumor diseases and fatty liver disease.

Therefore, in a specific embodiment, the method of the present invention relates to a method for predicting, preferably, in an apparently healthy subject, the risk of developing at least one sequela of the metabolic syndrome selected from the group consisting of tumor diseases, cardiovascular diseases, diabetes and fatty liver disease, comprising the steps of
(a) determining the amounts of adiponectin, retinol binding protein (RBP4) and proinsulin in a sample of said subject,
(b) comparing the amounts as determined in step a) to suitable reference amounts for adiponectin, RBP4 and proinsulin, and
(c) predicting the risk of developing at least one sequela of the metabolic syndrome.

The aforementioned method, preferably, further comprises the determination of the amount of a natriuretic peptide and/or CRP in a sample of said subject and comparing the, thus, determined amounts to a reference amount for a natriuretic peptide and/or CRP.

Preferred reference amounts are given herein above.

Diabetes according to the present invention, preferably, relates to type 2 diabetes. Definitions of diabetes mellitus are known to the person skilled in the art and diagnostic criteria have been established by the World Health Organization (WHO) in 1985 and 1999, as well as by the American Diabetes Association (ADA) in 1997. Any patient fulfilling the criteria according to one or more of these definitions is considered a type 2 diabetes patient. Preferably, the diabetes patient is defined according to the WHO 1999 criteria. Type 2 diabetes is also known as adult-onset diabetes or non-insulin-dependent diabetes mellitus (NIDDM). Type 2 diabetes can either be accompanied by adipositas (type 2a) or not be accompanied by adipositas (type 2b). Further types of diabetes are, e.g., caused by genetic defects, diseases of the exocrine pancreas, endocrinopathies, and influences of chemicals or pharmaceutical drugs. Subjects suffering from type 2 retain a certain level of endogenous insulin secretory function. However, insulin levels are low relative to the magnitude of insulin resistance and glucose levels. Type 2 diabetes, preferably, can be assessed by determining the fasting blood glucose level. A fasting blood glucose or serum glucose concentration greater than 125 mg/dL (6.94 mmol/L), preferably, indicates diabetes type 2. Moreover, type 2 diabetes can be assessed by carrying out the glucose tolerance test which is well known in the art. Preferably, a blood sugar level of 200 mg of glucose or larger per dL of plasma two hours after the intake of 75 g glucose (after over-night fasting) indicates type 2 diabetes. In a glucose tolerance test 75 g of glucose are administered orally to the patient being tested after 10-12 hours of fasting and the blood sugar level is recorded immediately before taking the glucose and 1 and 2 hours after taking it. How to determine blood glucose is well known in the art.

Tumor diseases are known in the art. Preferably, said tumor disease is selected from the group consisting of neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, particularly ERBB2 (erythroblastic leukemia viral oncogene homolog 2) positive breast cancer, urinary carcinoma, melanoma, brain tumors such as glioblastoma, astrocytoma, meningioma, medulloblastoma and peripheral neuroectodermal tumors, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma and plasmocytoma. A variety of cancer types are known in the art comprise neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeolid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma and plasmocytoma. More preferably, said tumor disease is selected from the group consisting of lung, colon, pancretic, gastric, prostate and ovarian cancer.

Fatty liver disease, frequently also referred to as hepatosteatosis is a prevalent liver condition that occurs when lipids accumulate in liver cells. The lipid accumulation may result in cellular injury. Fatty acid liver disease in the context of the present invention, preferably, is macrovesicular steatosis or microvesicular steatosis. Preferably, the term “fatty liver disease” as used herein relates to non-alcoholic fatty liver disease.

The term “cardiovascular disease” is known in the art. As used herein, the term, preferably, relates to any disorder or disease state associated with a structural or functional abnormality of the heart, or of the blood vessels supplying the heart, that impairs the normal functioning of the heart. Cardiovascular disease in the context of the present invention are, preferably, selected from the group consisting of coronary artery disease, atherosclerosis, myocardial infarction, cardiomyopathy, arteriosclerosis, hypertension, angina, and congestive heart failure.

Moreover, the present invention relates to a device for identifying a subject being susceptible to a metabolic syndrome related therapy comprising (i) means for determining the amount of adiponectin, RBP4 and proinsulin in a sample of a subject and (ii) means for comparing said amounts to suitable reference amounts, whereby a subject is identified which is susceptible to a metabolic syndrome related therapy.

Also envisaged by the present invention is a device for predicting, whether a subject which, preferably, is apparently healthy with respect to a metabolic syndrome is at risk of developing a metabolic syndrome (and/or a sequela of the metabolic syndrome) comprising (i) means for determining the amount of adiponectin, RBP4 and proinsulin in a sample of a subject and (ii) means for comparing said amounts to suitable reference amounts, whereby it is predicted whether said subject is at risk of developing a metabolic syndrome (and/or a sequela of the metabolic syndrome).

The term “device” as used herein relates to a system of means comprising at least the aforementioned means operatively linked to each other as to allow the prediction. Preferred means for determining the amount of proinsulin, RBP4, and adiponectin and means for carrying out the comparison are disclosed above in connection with the method of the invention. How to link the means in an operating manner will depend on the type of means included into the device. For example, where means for automatically determining the amount of the peptides are applied, the data obtained by said automatically operating means can be processed by, e.g., a computer program in order to obtain the desired results. Preferably, the means are comprised by a single device in such a case. Said device may accordingly include an analyzing unit for the measurement of the amount of the peptides or polypeptides in an applied sample and a computer unit for processing the resulting data for the evaluation. Alternatively, where means such as test strips are used for determining the amount of the peptides or polypeptides, the means for comparison may comprise control strips or tables allocating the determined amount to a reference amount. The test strips are, preferably, coupled to a ligand which specifically binds to the peptides or polypeptides referred to herein. The strip or device, preferably, comprises means for detection of the binding of said peptides or polypeptides to the said ligand. Preferred means for detection are disclosed in connection with embodiments relating to the method of the invention above. In such a case, the means are operatively linked in that the user of the system brings together the result of the determination of the amount and the prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test strips or electronic devices which merely require loading with a sample. The results may be given as output of raw data which need interpretation by the clinician. Preferably, the output of the device is, however, processed, i.e. evaluated, raw data the interpretation of which does not require a clinician. Further preferred devices comprise the analyzing units/devices (e.g., biosensors, arrays, solid supports coupled to ligands specifically recognizing the various markers as referred to herein, Plasmon surface resonace devices, NMR spectrometers, mass-spectrometers etc.) or evaluation units/devices referred to above in accordance with the method of the invention.

Furthermore, the present invention concerns a kit for carrying out the method for identifying a subject being susceptible to metabolic syndrome related therapy, said kit comprising instructions for carrying out the said method, and (i) means for determining the amount of adiponectin, RBP4 and proinsulin in a sample of a subject and (ii) means for comparing said amounts to suitable reference amounts, whereby a subject being susceptible to a metabolic syndrome related therapy is identified.

Also, the present invention envisages a kit for carrying out the method for predicting, in a subject which is, preferably, apparently healthy with respect to the metabolic syndrome, the risk of developing a metabolic syndrome (and/or a sequela of the metabolic syndrome) said kit comprising instructions for carrying out the said method, and (i) means for determining the amount of adiponectin, RBP4 and proinsulin in a sample of a subject which is apparently healthy with respect to a metabolic syndrome, and (ii) means for comparing said amounts to suitable reference amounts, whereby it is predicted whether said subject is at risk of developing a metabolic syndrome (and/or a sequela of the metabolic syndrome).

The term “kit” as used herein refers to a collection of the aforementioned means, preferably, provided separately or within a single container. The components of the kit may be comprised by separate vials (i.e. as a kit of separate parts) or provided in a single vial. Moreover, it is to be understood that the kit of the present invention is to be used for practising the methods referred to herein above. It is, preferably, envisaged that all components are provided in a ready-to-use manner for practising the methods referred to above. Further, the kit preferably contains instructions for carrying out the said methods. The instructions can be provided by a user's manual in paper- or electronic form. For example, the manual may comprise instructions for interpreting the results obtained when carrying out the aforementioned methods using the kit of the present invention.

It is to be understood that the aforementioned kits and devices according to the present invention, preferably, may further comprise means for determining the amount of a natriuretic peptide, particularly of NT-proBNP, and/or of CRP and means for comparing the amount of a natriuretic peptide, particularly of NT-proBNP, and/or of CRP to a suitable reference amount.

The following examples shall merely illustrate the invention. They shall not be construed, whatsoever, to limit the scope of the invention.

### Example 1

High molecular weight adiponectin, retinol binding protein 4, proinsulin, CRP and NT-proBNP were determined in serum samples obtained from a total of 2656 randomly selected subjects as well as in serum samples of 356 subjects with metabolic syndrome according to the European Group for the study of insulin resistance (EGIR, serum insulin larger 44 pmol/l, together with two of the following criteria: BMI larger than 30 kg/m², serum triglycerides larger 2 mmol/l or S-HDL lower 1 mmol/l, Glucose larger 6.1 mmol/l, blood pressure larger 140 systolic/90 diastolic mm Hg). HMW adiponectin was determined with the Adiponectin (Multimeric) EIA kit (Alpco Diagnostics, Salem, USA Catalog Number: 47-ADPH-9755), RBP4 was determined with the Retinol-binding protein(RBP)/RBP4 ELISA kit (Immundiagnostik, Bensheim, Germany, proinsulin was determined with the Human Intact Proinsulin ELISA kit (Biovendor GmbH, Heidelberg Germany Cat. No. RD193094100).

It was shown that there are statistically significant differences between the various biomarkers in patients with and without the metabolic syndrome (see Table 1)

TABLE 1
Significance analysis (Kruskall-Wallis) of biomarker levels
in patients with and without the metabolic syndrome

| Biomarker | p-Value |
|---|---|
| HMW Adiponectin | <0.001 |
| RBP4 | <0.001 |
| Proinsulin | <0.001 |
| NT-proBNP | <0.001 |

Generally, the amounts of proinsulin and RBP4 were larger in samples of individuals with the metabolic syndrome than in samples individuals without the metabolic syndrome, and the amount of adiponectin was lower in samples of individuals with the metabolic syndrome than in samples individuals without the metabolic syndrome. However, the data also showed that there are, on an individual basis, overlaps between the group of patients with metabolic syndrome and without metabolic syndrome. Thus, a portion of individuals without the metabolic syndrome had elevated proinsulin and RBP4 amounts and a reduced adiponectin amount indicating the need for a metabolic therapy. These are patients which will benefit from a metabolic syndrome related therapy since they are at risk of developing the metabolic syndrome and/or of suffering from a sequela thereof. Also, there were patients with the metabolic syndrome which, however, did not show an increase of amounts proinsulin and RBP4 and did not show a reduction of adiponectin. These are patients that will be overtreated with a therapy related to the metabolic syndrome (resulting, e.g., in increased health care costs).

Thus, the known criteria for the metabolic syndrome are not sufficient for assessing all metabolic disorders related to the metabolic syndrome. Therefore, subgroups of subjects with a metabolic syndrome may receive more treatment than required, whereas subgroups of subjects which are apparently healthy, but which require a certain metabolic syndrome related treatment, would not be treated when only applying the criteria for the metabolic syndrome.

The medians in the group of patients with the metabolic syndrome were 2.8 mg/l for CRP, 2.4 μg/l for adiponectin, 42.65 mg/l for RPB4 and 2.14 pmol/l for proinsulin. The medians for CRP, RBP4, and proinsulin were larger and the median for adiponectin was lower than the medians in the group of subjects being apparently healthy with respect to a metabolic syndrome (medians in group without metabolic syndrome: adiponectin 3.59 μg/ml; RBP4 37.11 mg/l; proinsulin 1.48 pmol/l; CRP 1.46 mg/l, see also FIG. 1).

However, some individuals in the group with apparently healthy subjects had increased amount of CRP, RBP4, NT-proBNP and proinsulin and decreased amounts of adiponectin indicating that these subject are increased risk of developing a metabolic syndrome or suffering from a sequela thereof. In the metabolic syndrome group, some individuals had normal levels of CRP, RBP4, proinsulin, adiponectin, and NT-proBNP (the same or nearly the same as the medians of the group of healthy subjects) indicating that these individuals are not at elevated risk of developing a sequela of the metabolic syndrome (although they suffer from the metabolic syndrome).

The method of the present invention allows a reliable identification of these subgroups.

### Example 2

A 52 year old male patient who has overweight (Body mass index: 30) and who fulfils the criteria for the metabolic syndrome (according to the ATP-III/NCEP criteria, slightly increased blood pressure, increased fasting plasma glucose according to an oral glucose tolerance test) presents at his family doctor. The patient wants to know whether it would be beneficial to reduce weight and to initiate a therapy with ACE inhibitors and statins. The biomarkers proinsulin (1.5 pmol/l), RBP-4 (38 mg/dl) and HMW adiponectin (3.1 μg/ml) are determined in a serum sample obtained from the patient. Based on these data, the doctor advises that the patient shall lose weight. However, a therapy with statins and ACE inhibitors shall not be started. After 6 months, the patient's body mass index is 30.5. Moreover, the adiponectin level has risen compared to the initial measurement and the RBP-4 and proinsulin levels remain unchanged.

### Example 3

A 56 year old male patient (Body mass index 26) with no known coronary artery disease presents at his family doctor. The patient does not have a metabolic syndrome as indicated by a normal blood pressure, fasting plasma glucose below 6.1 mmol/l (thus, not increased). Only the cholesterol level is slightly increased. The biomarkers proinsulin (3.2 pmol/l), RBP-4 (65 mg/di) and BMW adiponectin (2.75 μg/ml) are determined in a serum sample obtained from the patient. Based on these data, the physician recommends reducing the levels of cholesterol by reducing cholesterol in the daily diet.

After two years without any symptoms, the patient presents at his family doctor with urinary tract infection. Since urinary tract infections are frequently associated with diabetes type 2, fasting plasma glucose is determined. The results show that the patient has increased plasma glucose. Diabetes (type 2) is diagnosed.

What is claimed is:

1. A method for identifying susceptibility of a subject to a metabolic syndrome related therapy, wherein the subject has not developed a metabolic syndrome, the method comprising the steps of:
determining amounts of adiponectin, retinol binding protein 4 (RBP4), and proinsulin in a sample from the subject, and
identifying the subject's susceptibility to a metabolic syndrome related therapy by comparing the amounts of adiponectin, RBP4, and proinsulin determined to reference amounts for adiponectin, RBP4, and proinsulin, respectively.

2. The method of claim 1, wherein the metabolic syndrome related therapy is selected from the group consisting of angiotensin converting enzyme (ACE)-inhibitors, administration of diurectics, administration of lipid lowering drugs, administration of metformin hydrochloride, administration of thiazolidinediones, and administration of angiotensin-receptor blockers.

3. The method of claim 1, wherein (i) a determined amount of adiponectin lower than the reference amount for adiponectin, (ii) a determined amount of RBP4 greater than the reference amount for RBP4, and (iii) a determined amount of proinsulin greater than the reference amount for proinsulin indicates the subject's susceptibility to a metabolic syndrome related therapy.

4. The method of claim 1, wherein (i) a determined amount of adiponectin greater than the reference amount for adiponectin, (ii) a determined amount of RBP4 lower than the reference amount for RBP4, and (iii) a determined amount of proinsulin lower than the reference amount for proinsulin indicates that the subject is not susceptible to a metabolic syndrome related therapy.

5. The method of claim 1, wherein the reference amount for adiponectin is 2.8 μg/l and adiponectin is high molecular weight adiponectin, wherein the reference amount for RBP4 is 43 mg/l, and wherein the reference amount for proinsulin is 2.2 pmol/l.

6. The method of claim 1, wherein the sample is blood, plasma, or serum.

7. The method of claim 1 further comprising determining an amount(s) of a natriuretic peptide and/or C-reactive protein (CRP) in the sample and identifying the subject's susceptibility to a metabolic syndrome related therapy by additionally comparing the amount(s) of natriuretic peptide and/or CRP determined to a suitable reference amount (to suitable reference amounts) to reference amounts for the natriuretic peptide and/or CRP, respectively.

8. The method of claim 7, wherein a determined amount of the natriuretic peptide greater than the reference amount for NT-proBNP and/or a determined amount of CRP greater than the reference amount for CRP further indicates the subject's susceptibility to a metabolic syndrome related therapy.

9. The method of claim 7, wherein the natriuretic peptide is NT-proBNP, the reference amount for NT-proBNP is 100 pg/ml, and the reference amount for CRP is 4.0 mg/l.

10. A method for predicting a risk of developing a metabolic syndrome in a subject who is apparently healthy with respect to a metabolic syndrome, the method comprising the steps of:
determining amounts in a sample from the subject, and
predicting the subject's risk of developing a metabolic syndrome by comparing the determined amounts of adiponectin, retinol binding protein (RBP4), and proinsulin to reference amounts for adiponectin, RBP4, and proinsulin.

11. The method of claim 10, wherein (i) a determined amount of adiponectin lower than the reference amount for adiponectin, (ii) a determined amount of RBP4 greater than the reference amount for RBP4, and (iii) a determined amount of proinsulin greater than the reference amount for proinsulin indicates that said subject is at risk of developing a metabolic syndrome, and wherein (iv) a determined amount of adiponectin greater than the reference amount for adiponectin, (v) a determined amount of RBP4 lower than the reference amount for RBP4, and (vi) a determined amount of proinsulin lower than the reference amount for proinsulin indicates that the subject is not at risk of developing a metabolic syndrome.

12. A device for identifying, according to the method of claim 1, susceptibility of a subject to a metabolic syndrome related therapy, wherein the subject has not developed a metabolic syndrome comprising (i) means for determining amounts of adiponectin, RBP4, and proinsulin in a sample from a subject who has not developed a metabolic syndrome and (ii) means for comparing the amounts to reference amounts, whereby a subject is identified which is susceptible to a metabolic syndrome related therapy.

13. A kit for identifying, according to the method of claim 1, susceptibility of a subject to a metabolic syndrome related therapy, wherein the subject has not developed a metabolic syndrome, said kit comprising instructions for carrying out the method, and (i) means for determining the amount of adiponectin, RBP4, and proinsulin in a sample from the subject, and (ii) means for comparing the amounts to reference amounts, whereby a subject being susceptible to a metabolic syndrome related therapy is identified.
